(19) United States
(12) Patent Application Publication
(10) Pub. No.: US 2020/0168324 A1
(43) Pub. Date: May 28, 2020

(54) A MEDICAL IMAGING SYSTEM MANAGEMENT ARRANGEMENT

(71) Applicant: KONINKLIJKE PHILIPS N.V., EINDHOVEN (NL)

(72) Inventors: VINAY PARTHAN, BEST (NL); SATHISH KUMAR BALAKRISHNAN, BEST (NL)

(21) Appl. No.: 16/088,857

(22) PCT Filed: Apr. 4, 2017
(86) PCT No.: PCT/EP2017/058021

§ 371 Date: Sep. 27, 2018

(30) Foreign Application Priority Data
Apr. 4, 2016 (IN) 201641011781
May 17, 2016 (EP) 16169865.9

(51) Int. Cl. G16H 40/40; G16H 40/63; G06T 7/00

(52) U.S. Cl. CPC G16H 40/40; G16H 40/63; G06T 2207/30004; G06T 2207/30168; G06T 7/0012

(57) ABSTRACT

It is an object of the invention to provide for a system than can help in using medical imaging systems in an improved way by at least one of reducing operating costs of the medical imaging systems or by improving image quality. This object is achieved by a medical imaging system management arrangement comprising one or more medical imaging systems configured to acquire medical imaging data, wherein the medical imaging systems have a current performance and/or an expected future performance. The medical imaging system management arrangement further comprises a central unit connected to the one or more medical imaging systems via a data link. The one or more medical imaging systems are configured to send performance data related to the current and/or the expected future performance to the central unit, wherein the central unit further comprises an analyzer configured to analyze the performance data and provide it to a user.

### FIELD OF THE INVENTION

The invention relates to the field of diagnostic imaging and more specifically to the field of magnetic resonance imaging.

### BACKGROUND OF THE INVENTION

US2003/0181804 describes a plurality of diagnostic scanners that share access to a remote communal processing center that performs reconstruction and post reconstruction processing for various modalities. Each of the diagnostic scanners submits a data set to the remote center electronically over the lines. The reconstructed image representations are sent electronically to the address that sent them.

### SUMMARY OF THE INVENTION

It is an object of the invention to provide for a system than can help in using medical imaging systems in an improved way by at least one of reducing operating costs of the medical imaging systems or by improving image quality. This object is achieved by a medical imaging system management arrangement according to claim 1.

Inefficient use or down time of a medical imaging system can have a large influence on the operating costs of a medical imaging system. By keeping track of and by analyzing current and/or expected future performance data, insight can be obtained in causes of inefficient use and/or downtime. The invention is especially advantageous if the central unit is connected to multiple medical imaging systems, because in that way the current and/or expected future performance data between different medical imaging systems can be easily compared.

Inefficiency in the use of medical imaging systems can have several causes. One of these causes is that due to insufficient image quality rescans are needed. By keeping track of current and/or future performance data, image quality may be improved as will be discussed below.

According to embodiments of the invention, one or more medical imaging system is a magnetic resonance imaging (MRI) system.

According to one embodiment of the invention the performance data comprises information about at least one out of image quality per medical imaging system and/or per medical image and/or efficiency per medical imaging system. Efficiency could for example be determined based on exams per day, patient setup times, number of rescans needed, actual acquisition times, coil positioning times, time that the medical imaging system is unused, inter scan delay, inter exam delay. In this way it can be determined if a certain medical imaging system underperforms in terms of image quality. Also it may be detected if efficiency, at a certain medical imaging system is low compared to other medical imaging systems. Similarly, it may be determined if a certain medical imaging system acquires images which image quality is above average or even outstanding compared to images acquired with other medical imaging systems. Additionally, it may be detected that a certain medical imaging system is used more efficiently than others. This embodiment is advantageous, because such diagnosis of under or over performance may be a first step in finding the cause of inefficient use of insufficient image quality.

Insufficient image quality could be determined e.g. by means of determination of the signal to noise ratio (SNR) of an image, the contrast to noise ratio (CNR) of an image, presence of artifacts, visibility of relevant anatomical features, success of fat suppression (in the case of MRI), by the number of times a rescan is needed. One or more of these measures related to image quality could be related to potential causes like e.g. transmit and/or receive coil used (in case of MRI), presence or absence of certain software upgrade, imaging sequence used (in case of MRI), imaging parameters used, scan time, performance decrease of the medical imaging system or one or more of its components. By comparing the measures related to image quality with the potential causes both for images with above average quality and for images with below average quality, recommendations can be determined on how to improve image quality in certain cases. Instead of doing this on an image level, the analysis can also be performed on an imaging system level. By comparing the measures related to image quality with the potential causes both for medical imaging systems delivering images with above average quality and for medical imaging systems delivering images with below average quality, it can be determined what factors affect image quality. Based on that recommendations can be made on how to improve image quality for under performing medical imaging systems.

According to further embodiments of the invention the medical imaging system management arrangement is configured to provide recommendations to a user on how to improve image quality for under performing medical imaging systems. This is advantageous as it may reduce the number of rescans required and may thereby improve efficiency. Examples of recommendations could be use of a different imaging sequence, replacement of a transmit and/or receive coil, use of a different transmit and/or receive coil, different selection of imaging parameters.

According to further embodiments of the invention, the performance data comprises information about at least one out of number of exams per day, patient setup times, number of rescans needed, actual acquisition times, coil positioning times, time that the medical imaging system is unused, inter scan delay, inter exam delay. Performance data could also comprise Patient Change Over Time, Patient Preparation Time. An Interscan delay could be due to a technician doing some activities like filming, post processing etc (or) due to inadequate training of the technician. This embodiment is advantageous, because by obtaining insight in where time is spend it can be more easily determined how efficiency can be improved.

According to further embodiments of the invention the at least one out of number of exams per day, patient setup times, number of rescans needed, actual acquisition times, coil positioning times, time that the medical imaging system is unused, inter scan delay, inter exam delay are compared between medical imaging systems. In this way it can be determined if certain medical imaging systems are under or overperforming in terms of efficiency.

According to further embodiments of the invention the medical imaging system management arrangement is configured to relate for the multiple medical imaging systems the performance data to potential causes. This is advantageous, because in this way solutions to insufficient efficiency can be provided. Potential causes could be for example the anatomical site from which images are acquired or a amount of variation in the anatomical sites from which images are acquired. Other causes could be exam type used and/or whether the exam type is related to the anatomical site that is being imaged. Other causes could be related to the coils used, the sequence used, a current operator and corresponding skill level or workflow related aspects.

According to further embodiments of the invention the medical imaging system management arrangement is further configured to determine at least one out of an improved imaging schedule, imaging protocol or workflow for one or more of the medical imaging systems based on the performance data and provide this to the user. Recommendations could include to redistribute patients over the medical imaging systems such that similar anatomical sites are acquired at a single medical imaging system. Other recommendations could be to redistribute operators such that less skilled operator will have to deal with a reduced number of complex acquisitions and/or preparations. Other recommendations could be to study the workflows that have shown to lead to a better efficiency or to use a different acquisition sequence and/or coil.

According to further embodiments of the invention the central unit is configured to at least partly control the one or more medical imaging systems. This embodiment is advantageous, because especially at certain locations skilled personnel that is able to control the medical imaging systems is scarce. By allowing system control from the location of the central unit the number of skilled persons needed to control the medical imaging systems may be reduced. For example, in current clinical practise, many medical imaging systems are operated by two people. During certain parts of an imaging procedure there is relatively a lot of work (e.g. when setting up the patient). However, during other times there is less work to be done (e.g. during actual image acquisition). By moving part of the control of the medical imaging system to the location of the central unit, one may be able to reduce the amount of skilled personnel needed.

According to further embodiments of the invention, the medical imaging system management arrangement comprises multiple medical imaging systems, wherein the medical imaging systems operate according to an imaging schedule. The imaging schedule comprises first periods and second periods, wherein workload for an operator is lower during the first periods than during the second periods. The medical imaging system management arrangement is configured to generate an imaging schedule per medical imaging system such that overlap in second periods between different medical imaging systems is reduced or limited. This embodiment is advantageous, because certain parts of the imaging procedure result in more work load than others. By desynchronizing the time points related to high workload, peaks in workload at the location of the central unit can be reduced. This is advantageous, because in this way efficiency may be improved and waiting times at the locations of the medical imaging systems may be reduced. For this embodiment it is not essential that wherein the central unit further comprises an analyzer configured to analyze the performance data and provide it to a user.

According to further embodiments of the invention the medical imaging systems comprises one or more components and the performance data comprises information about a measure for a current performance and/or expected future performance of the one or more components. This embodiments is advantageous, because it may help in early diagnosis of potential component failure.

According to further embodiments of the invention, the medical imaging system management arrangement is configured to relate the measure for the current performance and/or expected future performance from a previous date to actual data about component breakdown or performance decrease and wherein medical imaging system management arrangement is further configured to use this relation for improved prediction of performance decrease and/or component breakdown. This embodiment is advantageous because in this way prediction of performance decrease and/or component breakdown may be improved.

According to a further embodiment of the invention the medical imaging system management arrangement is configured such that a current or predicted future performance decrease can be resolved from the central unit. This is advantageous because in this way costs may be reduced. For this embodiment it is not essential that wherein the central unit further comprises an analyzer configured to analyze the performance data and provide it to a user.

According to other aspects, the invention is an analyzer configured to be used in a medical imaging system management arrangement as discussed above.

These and other aspects of the invention will be apparent from and elucidated with reference to the embodiments described hereinafter.

### BRIEF DESCRIPTION OF THE DRAWINGS

FIG. 1 diagrammatically shows a medical imaging system management arrangement according to embodiments of the invention and

FIG. 2 diagrammatically shows an example of performance data and

FIG. 3 diagrammatically shows another example of performance data and

FIG. 4 diagrammatically shows another example of performance data and

FIG. 5 diagrammatically shows an overview of anatomical sites scanned and

FIG. 6 diagrammatically shows average exam times and average preparation, finish and idle times for different anatomical sites and

FIG. 7 shows an overview of sequences used per anatomical site and

FIG. 8 diagrammatically shows an overview of imaging schedules for multiple MRI systems and

FIG. 9 diagrammatically shows a medical imaging system management arrangement configured for detecting insufficient image quality of a scan based on comparison of the image quality of the scan to image quality data provided by the multiple medical imaging systems

FIG. 10 diagrammatically shows a magnetic resonance imaging system.

### DETAILED DESCRIPTION OF EMBODIMENTS

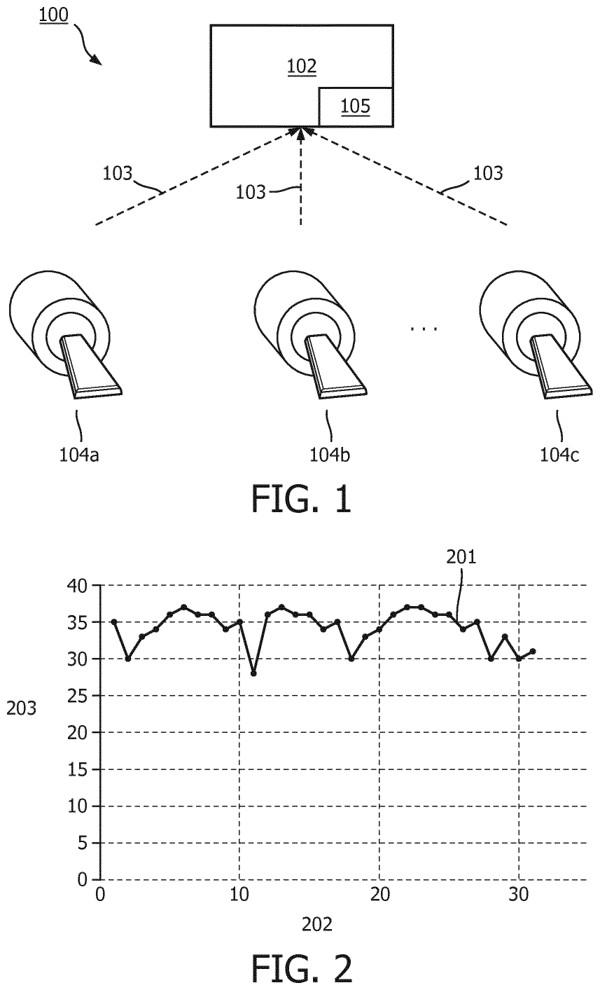

FIG. 1 diagrammatically shows a medical imaging system management arrangement according 100 to embodiments of the invention. The medical imaging system management arrangement comprises multiple MRI systems 104a, 104b, 104c. FIG. 1 only shows three MRI systems, but there could be many more. The medical imaging system management arrangement further comprises a central unit 102 connected to the one or more medical imaging systems via a data link 103. The MRI systems 104a, 104b, 104c are configured to send performance data 201, 301, 401, 601, 901 related to the current and/or the expected future performance to the central unit 102. The central unit 102 comprises an analyzer 105 configured to analyze the performance data and provide it to a user. The medical imaging system management arrangement is configured such that the one or more medical imaging systems can at least partly be controlled by the central unit.

FIG. 2 diagrammatically shows an example of performance data 201. In this figure a number of exams performed per day 201 are shown for a certain MRI system 104a. The x-axis 202 shows the days (31 in total) and the y-axis 203 shows the number of exams. The minimum number of exams per day was 28 over the time period of 31 days. The maximum was 37 and the average was 28. The analyzer is configured to compare these numbers to performance data from other MRI systems, e.g. to data from 104b and/or data from 104c. In this way it can be determined if the performance of system 104a is sufficient or if there may be overperformance or underperformance.

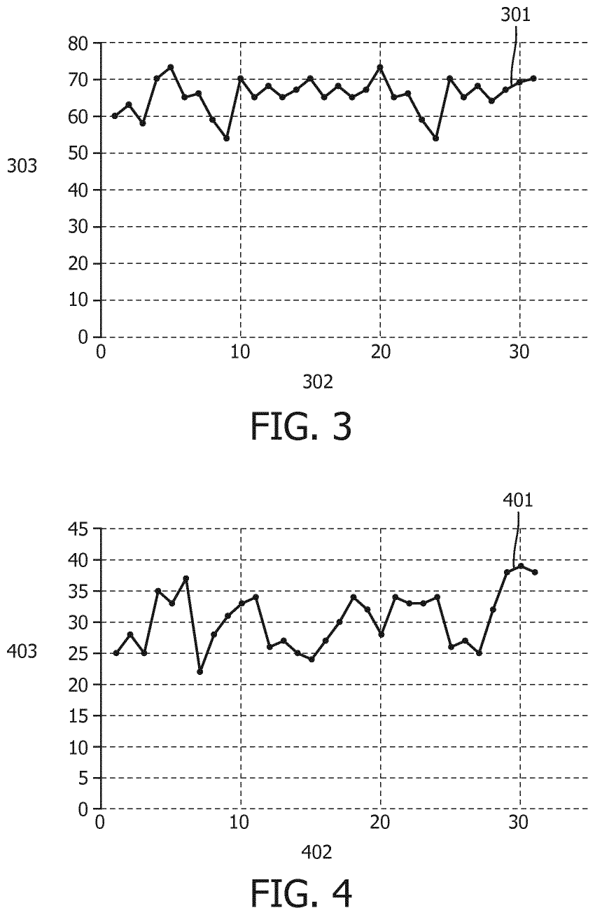

FIG. 3 diagrammatically shows an example of performance data 301. In this figure an exam efficiency per day 301 is shown for a certain MRI system 104a. The (exam) efficiency is the exam time per patient divided by the procedure time per patient. Here the exam time for a single patient is defined as the total time needed for scanning and the inter scan delay(s). The procedure time is the total of the exam time and the idle time and the time needed for patient preparation and finishing. This is only one way of determining an exam efficiency. Alternatives are possible and will be obvious to the skilled person. In FIG. 3, the x-axis 302 shows the days (31 in total) and the y-axis 303 shows the exam efficiency. The average exam efficiency is 65%. The analyzer can compare this number to performance data from other MRI systems, e.g. to data from 104b and/or data from 104c. In this way it can be determined if the performance of system 104a is sufficient or if there may be overperformance or underperformance.

FIG. 4 diagrammatically shows an example of performance data 401. In this figure a scan efficiency per day 401 is shown for a certain MRI system 104a. The (scan) efficiency is the scan time per patient divided by the procedure time per patient. Here the scan time for a single patient is defined as the total time needed for scanning without the inter scan delay(s). The procedure time is the total of the exam time and the idle time and the time needed for patient preparation and finishing. This is only one way of determining a scan efficiency. Alternatives are possible and will be obvious to the skilled person. The x-axis 302 shows the days (31 in total) and the y-axis 303 shows the scan efficiency. The scan efficiency is 30%. The analyzer is configured to compare these numbers to performance data from other MRI systems, e.g. to data from 104b and/or data from 104c. In this way it can be determined if the performance of system 104a is sufficient or if there may be overperformance or underperformance.

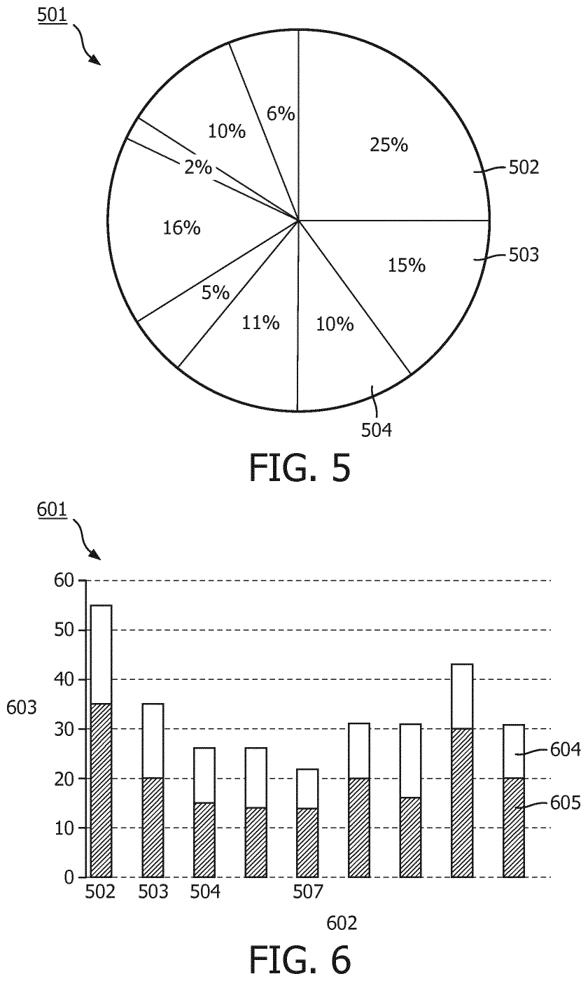

FIG. 5 diagrammatically shows an overview of anatomical sites scanned 501. Examples of anatomical sites are the head 502, knee 503 and shoulder 504. Exam efficiency and/or scan efficiency could be related to the anatomical site scanned. Also, a (large) variation in anatomical sites scanned can have an effect on the exam and/or scan efficiency. By relating the information about anatomical site scanned to the performance data, preferably over multiple MRI systems, it can be determined if the anatomical site scanned and/or the variation in anatomical sites scanned could be a cause for the performance data of a certain MRI system 104a. The medical imaging system management arrangement may recommend to distribute patients over the MRI systems such that the number of anatomical sites scanned per MRI system is reduced.

FIG. 6 diagrammatically shows average exam times (dark areas, 605) and average preparation, finish and idle times (light areas, 604) for different anatomical sites 602 for MRI system 104a. Examples of anatomical sites could be head 502, knee 503, shoulder 504 and prostate 507. Y-axis 603 shows time in minutes. Average exam times and average preparation, finish and idle times can be compared for the different anatomical sites. It can be seen that the average preparation, finish and idle times for head scans are relatively large compared to average preparation, finish and idle times for other anatomical sites. Additionally or alternatively average exam times and average preparation, finish and idle times for the different anatomical sites for MRI system 104a can be compared to the same measures for other MRI systems 104b, 104c. For example average preparation, finish and idle times may be high for head scans on MRI system 104a compared to the other MRI systems 104b, 104c. It may therefore be worthwhile to compare workflow related aspects of MRI system 104a to workflow related aspects of MRI systems 104b, 104c. In this way the workflow and/or the efficiency at 104a may be improved. On the other hand average preparation, finish and idle times for prostate 507 may be low for MRI system 104a. It may therefore be worthwhile to compare workflow related aspects of MRI system 104a to workflow related aspects of MRI systems 104b, 104c. In this way the workflow and/or the efficiency at 104b and 104c may be improved. The medical imaging system management arrangement may be configured to recommend to look further into specific workflow related aspects and to compare those to the workflows of (preferably nearby) well performing systems.

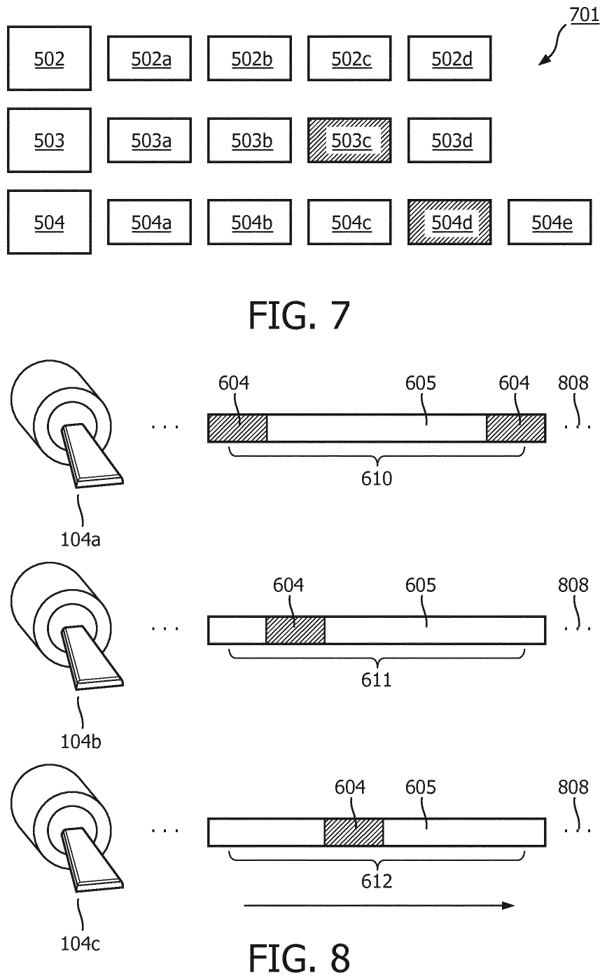

FIG. 7 shows an overview of sequences 502a, b, c, d, 503a, b, c, d, 504a, b, c, d used per anatomical site 502, 503, 504. Sequences 503c and 504d are highlighted because they are not considered to be optimized for use in scanning anatomical sites 503 and 504 respectively. As a result those sequences may result in insufficient image quality and/or reduced efficiency. Therefore, the medical imaging system management arrangement may be configured to recommend to an operator of the MRI system 104a to use an alternative imaging sequence.

FIG. 8 diagrammatically shows an overview of imaging schedules 610, 611, 612 for multiple MRI systems 104a, 104b, 104c. Each imaging schedule comprises first periods 605 and second periods 604. A lead technician can at least partly control the one or more MRI systems from the location of the central unit. This is advantageous, because in this way less personel is needed to operate the MRI systems. The workload at the central location is higher during second periods 604 than during first periods 605.

It is advantageous if peaks in workload (e.g. when preparing for a new patient. However this could also be other periods) for the multiple MRI systems do not come at the same time for the lead technician, because this could result in unnecessary delays at the locations of the MRI systems, e.g. an operator of a second MRI system may need to wait until the lead technician is finished with the preparations for a first MRI system, which will reduce efficiency. To solve this issue the medical imaging system management arrangement is configured to generate an imaging schedule per medical imaging system such that overlap in second periods 604 between different medical imaging systems is reduced or limited. An example of this is displayed in FIG. 8.

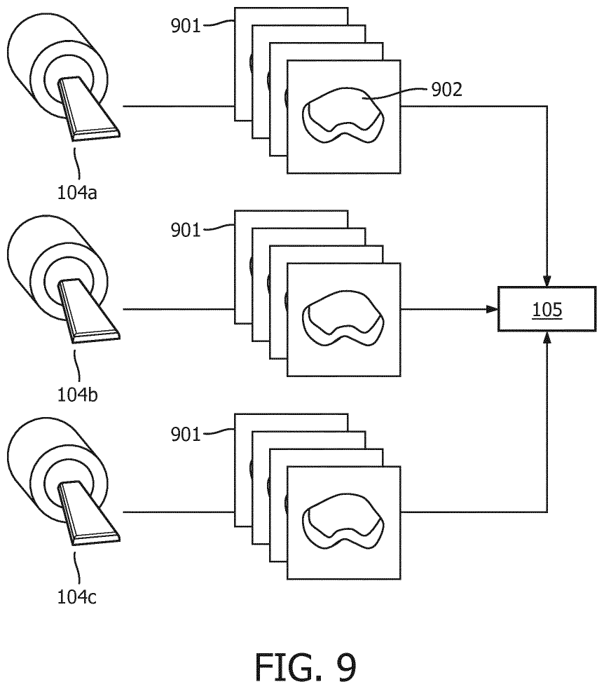

FIG. 9 diagrammatically shows a medical imaging system management arrangement configured for detecting insufficient image quality of a medical image based on comparison of the image quality of the medical image to image quality data provided by the multiple medical imaging systems 104a, 104b, 104c. The medical imaging systems are configured to send performance data 901 to the analyzer 105. The performance data in this figure are T2w MRI images of the prostate 902. However, they could be any other medical images. Also, the performance data could be measures reflecting image quality, like e.g. signal to noise ratio (SNR) of an image, the contrast to noise ration (CNR) of an image, presence of artifacts, visibility of relevant anatomical features, success of fat suppression (in the case of MRI), by the number of times a rescan is needed. Both the medical images themselves and the measures reflecting image quality are considered image quality data. The analyzer could be configured to retrieve the measures reflecting image quality from the medical images by known image processing methods. The analyzer 105 is further configured to compare the measures reflecting image quality from different scans and/or different systems. In this way the analyzer is configured to determine if a certain medical image (e.g. T2w prostate 902) shows insufficient image quality. The medical imaging system management arrangement is configured to relate the image quality to potential causes for the multiple medical imaging systems. These causes could be transmit and/or receive coil used (in case of MRI), imaging sequence used (in case of MRI), imaging parameters used, scan time. Especially, when a lot of data is available, the medical imaging system management arrangement may be able to relatively easily determine a cause of insufficient image quality. When this cause has been determined the medical imaging system management arrangement may recommend to for example change one of the transmit and/or receive coil used (in case of MRI), imaging sequence used (in case of MRI), one or more imaging parameters used, scan time. This recommendation could be based on the measures which were used during acquisitions which led to sufficient image quality.

The medical imaging system management arrangement could also be configured to combine the analyses on efficiency and image quality, such that recommendations can be made which balance efficiency and image quality.

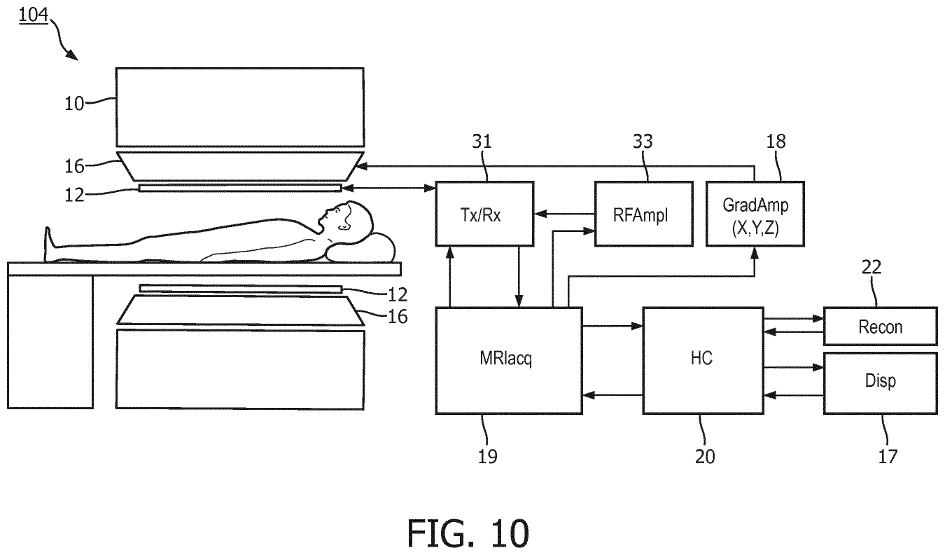

The medical imaging systems 104a, 104b, 104c comprise one or more components. Examples of such components are diagrammatically shown in FIG. 10 and could be RF amplifiers 33, gradient amplifiers 18, RF system 12, gradient system 16, Tx/Rx Switch 31, magnet 10. The performance data comprises information about a measure for a current performance and/or expected future performance of the one or more components. From the location of the central unit 102, one can look into certain parameters that are logged realtime in the system (For eg., Helium pressure or Cold Head pressure or Chiller flow velocity etc), Those parameters can be used in a predictive model to understand the magnet performance to predict the future downtime. Similarly, parameters of RF Amplifiers, Gradient Amplifiers can be constantly plotted to see the variance in performance to predict the future downtime. Similarly, a constant degrade of SNR from a particular coil could be used to predict the coil failure earlier.

If a parameter (or) certain set of parameters can be remotely monitored at regular intervals, it can help predict failures of hardware or software components. For eg., if we can keep monitoring some parameters like Rise/Fall time, Rising/Falling edge overshoot, Pulse overshoot, Amplitude/Phase stability over a period of time, it may help in predicting the performance of the RF Amplifier. When a downward trend of the performance is seen, the service engineers can be informed immediately to rectify the issue (or) replace the amplifier proactively, before even it goes down, so that the users of the medical imaging systems will not be negativly affected by a down-time of the system.

Alternatively or additionally the memory profile can be monitored of a particular software process in the system and it is recognized that this goes out of control, this can be handled remotely by having a process that can take care of the memory usage on the medical imaging system.

Whilst the invention has been illustrated and described in detail in the drawings and foregoing description, such illustrations and description are to be considered illustrative or exemplary and not restrictive; the invention is not limited to the disclosed embodiments.

1. A medical imaging system management arrangement comprising:
multiple medical imaging systems configured to acquire medical imaging data, wherein the medical imaging systems have a current performance and/or an expected future performance and wherein the medical imaging systems are suitable to be used to acquire imaging data from multiple subjects and wherein the performance data comprises information about at least one out of image quality per medical image and/or per medical imaging system;
a central unit connected to the one or more medical imaging systems via a data link,
wherein the one or more medical imaging systems are configured to send performance data related to the current and/or the expected future performance to the central unit, wherein the central unit further comprises an analyzer configured to analyze the performance data and provide it to a user,
wherein the medical imaging system management arrangement is further configured for detecting insufficient image quality of a medical image based on comparison of an image quality of the medical image to image quality data provided by one or more of the multiple medical imaging systems.

2. The medical imaging system management arrangement of claim 1, wherein one or more of the medical imaging systems is an MRI system.

3. The medical imaging system management arrangement of claim 1, configured to relate for the multiple medical imaging systems the image quality to potential causes.

4. The medical imaging system management arrangement of claim 1, wherein the performance data comprises information about at least one out of number of exams per day, patient setup times, number of rescans needed, actual acquisition times, coil positioning times, time that the medical imaging system is unused, inter scan delay, inter exam delay.

5. The medical imaging system management arrangement of claim 4, configured to compare the at least one out of exams per day, patient setup times, number of rescans needed, actual acquisition times, coil positioning times, time that the medical imaging system is unused, inter scan delay, inter exam delay between the medical imaging systems

6. The medical imaging system management arrangement of claim 5, configured to relate for the multiple medical imaging systems the performance data to potential causes.

7. The medical imaging system management arrangement of claim 1, further configured to determine at least one out of an improved imaging schedule, imaging protocol or workflow for one or more of the medical imaging systems based on the performance data and provide this to the user.

8. The medical imaging system management arrangement of claim 1, wherein the central unit is configured to at least partly control the one or more medical imaging systems.

9. The medical imaging system management arrangement of claim 8, wherein the medical imaging systems operate according to an imaging schedule, wherein the imaging schedule comprises first periods and second periods, wherein workload for an operator is lower during the first periods than during the second periods and wherein the medical imaging system management arrangement is configured to generate an imaging schedule per medical imaging system such that overlap in second periods between different medical imaging systems is reduced or limited.

10. The medical imaging system management arrangement of claim 1, wherein the medical imaging systems comprise one or more components and wherein the performance data comprises information about a measure for a current performance and/or expected future performance of the one or more components.

11. The medical imaging system management arrangement of claim 10, wherein the medical imaging system management arrangement is configured to relate the measure for the current performance and/or expected future performance from a previous date to actual data about component breakdown or performance decrease and wherein medical imaging system management arrangement is further configured to use this relation for improved prediction of performance decrease and/or component breakdown.

12. An analyzer configured to be used in a medical imaging system management arrangement as claimed in claim 1.
